(19) United States
(12) Patent Application Publication
(10) Pub. No.: US 2015/0299431 A1
(43) Pub. Date: Oct. 22, 2015

(54) METHOD FOR THE REACTIVE EXTRUSION OF AN AMYLACEOUS MATERIAL IN THE PRESENCE OF A POLYPHOSPHATE SERVING AS A CROSS-LINKING AGENT, RESULTING PRODUCTS, AND USES THEREOF

(71) Applicant: ROQUETTE FRERES, Lestrem (FR)

(72) Inventors: Julien PARCQ, Lille (FR); Rene SAINT-LOUP, Lomme (FR); Vincent WIATZ, Lomme (FR)

(21) Appl. No.: 14/418,529

(22) PCT Filed: Jul. 30, 2013
(86) PCT No.: PCT/FR2013/051831

§ 371 Date: Jan. 30, 2015

(30) Foreign Application Priority Data
Jul. 31, 2012 (FR) 12 57436

(51) Int. Cl. C08L 3/02; C08L 101/00; C08B 31/00; A23L 1/00; A61K 47/36; A61K 8/73; A01N 25/12; A23L 1/0522; D21H 17/28; C09D 103/04

(52) U.S. Cl. CPC C08L 3/02; D21H 17/28; C08L 101/00; C08B 31/003; C09D 103/04; A61K 47/36; A61K 8/732; A01N 25/12; A23L 1/0522; A23L 1/0076; A61K 2800/10; A23V 2002/00

(57) ABSTRACT

A method for the reactive extrusion of an amylaceous substance in the presence of a polyphosphate serving as a cross-linking agent, to the resulting products, and to the uses thereof. In particular, a method for the reactive extrusion of starch in the presence of a cross-linking agent which is a polyphosphate, in particular sodium trimetaphosphate, which behaves like an effective substitute for glyoxal. The method makes it possible to manage the competition between the destructuring and cross-linking mechanisms of the amylaceous substance. The crystalline phase content of the cross-linked starches is thus controlled in accordance with the specific needs of the final use.

The present invention relates to a method for the reactive extrusion of amylaceous material in the presence of a crosslinking agent which is a polyphosphate and more preferably sodium trimetaphosphate. Sodium trimetaphosphate behaves as an effective substitute for the glyoxal used and recommended by the prior art. Thus, in some of its embodiments the method according to the invention advantageously makes it possible to control the competition between the destructuring and the crosslinking of the starch. This method thus makes it possible to obtain crosslinked starches with contents of residual crystalline phases which are adaptable in order to respond ideally to the specific requirements of different final applications.

Reactive extrusion is a well-known technique for shaping amylaceous materials in the form of particles of nanometer size, which can then be dispersed in water or in an aqueous alcoholic solvent. This technology is based on a first stage of extrusion of the amylaceous material in the presence of a crosslinking agent, followed by a granulation stage and by grinding. By addition of water or an aqueous alcoholic solvent, it is possible finally to create dispersions with a solid matter content at least equal to 20% dry weight of amylaceous material, stable over time, and exhibiting a particle size of between 100 and 500 nm as determined by laser particle size analysis.

This technology was described in the documents EP 1 159 301, then recapitulated and refined in the documents EP 1 303 667, EP 1 303 670 and EP 2 251 484, these 3 documents targeting applications in paper manufacture for the products obtained. More precisely, these latter documents are directed at the manufacture of compositions intended to cover the sheet of paper in order in particular to endow it with improved optical properties, said compositions being known to those skilled in the art under the expression “coating colors”.

As the last 3 documents cited demonstrate, the utilization of particles made by reactive extrusion in paper coating colors makes it possible partially to replace the synthetic binders currently used in such applications, and this while maintaining an equivalent level of properties: both in the coating color itself, in terms of high and low shear gradient viscosity (Brookfield and Haake viscosity), but also in sheets of paper in terms of water retention and printability.

These documents contain a disclosure very much based on the crosslinking agent which appears as an essential component of the extrusion stage. This crosslinking agent, which is present within the extruder and will therefore react with the amylaceous material within the extruder, can be introduced, added to the starch just before being introduced into the extruder (premix) and/or introduced directly into the extruder, preferably directly into the extruder. The crosslinking agent may or may not be reversible. In the category of reversible crosslinking agents, these documents cite the polyaldehydes and in particular dialdehydes such as glutaraldehyde and glyoxal, and carbohydrates, glyoxal being most preferred. Among the irreversible crosslinking agents, epichlorohydrin may be mentioned. It is noted that only glyoxal is illustrated in the examples, without any proof that the other agents mentioned function just as well in the method described.

Now glyoxal has disadvantages in quite a few respects. It is most often delivered in the form of very acidic and therefore corrosive aqueous solutions (pH close to 2). Moreover, it is an extremely reactive compound which can react violently with oxidizing agents, acids and strong bases. Further, like all aldehydes, it is strongly irritant to the skin and mucous membranes. Finally, glyoxal is a product described as “CMR”: mutagenic and toxic for reproduction. It is labeled H341, which signifies that it is liable to induce genetic abnormalities.

Consequently, there is an unsolved technical problem, consisting in finding a crosslinking agent capable of effectively replacing glyoxal in a reactive extrusion process.

Working with this aim, the Applicant has succeeded in demonstrating that polyphosphates and in particular sodium trimetaphosphate meet these requirements.

Also, a first subject of the present invention consists in a method for manufacture of particles consisting of at least one amylaceous material, said method comprising:
a) at least one stage of extrusion of at least one amylaceous material, in the presence of at least one crosslinking agent, b) a granulation stage,
c) possibly a grinding stage, and
d) possibly a stage of dispersion in a solvent.
   characterized in that the crosslinking agent is a polyphosphate.

The document WO 2004/085481 discloses a method for production of particles of an amylaceous material by extrusion, said extrusion taking place on a previously crosslinked amylaceous material. In fact, it is stipulated in particular on page 4 lines 24 to 28 that initially a paste is formed by mixing between starch, water, an alkali and a crosslinking agent and that said paste is then introduced into an extruder.

The article by Bi Zheng Li and coworkers, Journal of Food Engineering 92 (2009) 255-260, discloses a method for production of particles of an amylaceous material crosslinked with sodium trimetaphosphate. This article in no way discloses an extrusion stage or any treatment or kneading of amylaceous material under strong shear stresses in the presence of sodium trimetaphosphate.

More particularly, the method which is the subject of the present invention consists in a method for production of particles consisting of at least one amylaceous material, said method comprising:
a) at least one stage of extrusion of at least one amylaceous material, in the presence of at least one crosslinking agent, b) a granulation stage,
c) possibly a grinding stage, and
d) possibly a stage of dispersion in a solvent,
   characterized in that the crosslinking agent is a polyphosphate and in that the extrusion stage a) is performed by introduction of the amylaceous material, polyphosphate and a solvent into the extruder.

In a preferred embodiment of the method which is the subject of the present invention, the polyphosphate is sodium trimetaphosphate.

The extrusion stage a) of the method according to the invention is performed in an extruder: this device is the site of substantial shear forces which are applied to the starch or to the amylaceous material. Further, this stage is performed at a temperature at least equal to 40° C., preferably at least 50° C., very preferably at least 60° C., but in all cases at a temperature lower than the degradation temperature of the amylaceous material; the selection of this temperature falls within the normal skills of those skilled in the art, with regard to the physical and chemical characteristics of the materials used. The method generates a pressure of between 5 bars and 150 bars.

More particularly, the extrusion stage a) is performed by introduction of the non-crosslinked amylaceous material and the polyphosphate into the extruder.

According to the method which is the subject of the invention, the amylaceous material or starch is subjected to substantial shear forces in the presence of the crosslinking agent in an extruder. The crosslinking of the starch thus takes place within the extruder.

More particularly, the method which is the subject of the invention thus consists in a method for production of particles consisting of at least one crosslinked amylaceous material, said method comprising:
a) at least one stage of extrusion of at least one amylaceous material, in the presence of at least one crosslinking agent, b) a granulation stage,
   the crosslinking agent being a polyphosphate and the extrusion stage comprising:
i) a stage of introduction of the amylaceous material, the crosslinking agent and a solvent into an extruder and
ii) a stage of kneading of the amylaceous material under strong shear in the presence of the crosslinking agent.

The non-crosslinked amylaceous material is generally introduced into the first zones of the extruder, into zone 1 or at the foot of the extruder, but can equally be introduced into any zone except for the last one. This introduction can be effected by gravimetric insertion via the top of the extruder or by use of specific introduction systems known to those skilled in the art for example as “side-feeders”.

The amylaceous material can be introduced mixed with another amylaceous material and/or mixed with another constituent different from an amylaceous material. As an example of another constituent different from an amylaceous material, antimicrobial agents, plasticizers other than water such as polyols (e.g. ethylene glycol, propylene glycol, glycerol or maltose), urea, sodium lactate, etc. may be mentioned. This mixture is thus generally introduced into zone 1, but can also be introduced into any zone of the extruder except for the last. For this purpose, the mixture can be obtained by homogenization in a device of the “dry-blend” type.

Likewise or according to another embodiment, the amylaceous material can be introduced in combination with another amylaceous material and/or with another constituent different from an amylaceous material, not in the form of a mixture but separately. According to this embodiment, the ingredients are introduced separately either into the same zone (into any zone except for the last zone but preferably into the first zones of the extruder or at the foot), or into distinct zones.

In all cases, when the ingredients are introduced separately, they are introduced from standard metering systems well known to those skilled in the art.

The extrusion stage a) of at least one amylaceous material is performed, as well as by introduction into the extruder of the amylaceous material to be crosslinked and of the crosslinking agent, in this case the polyphosphate, by introduction into the extruder of at least one solvent, preferably of one solvent. The extrusion in fact takes place in a solvent medium.

Thus the extrusion stage a) is performed by introduction of at least one solvent selected from water and aqueous alcoholic solvents into the extruder. Preferably it is water. This solvent or mixture of solvents is used to destructure the amylaceous fraction. In other words, the solvent in particular can act and does act as a plasticizer.

The solvent or mixture of solvents can be introduced into any zone of the extruder.

Thus the solvent or solvent mixture can be introduced after the amylaceous material to be crosslinked and the crosslinking agent (the polyphosphate) have been introduced into the extruder.

The further towards the end of the extruder the solvent or mixture of solvents is added, the more the duration of the destructuring of the starch will be reduced and hence the less will be the degree of destructuring of the amylaceous material or starch.

It is also possible to envisage systems where the solvent is injected before the introduction of the amylaceous material into the extruder.

Still according to another embodiment of the method which is the subject of the invention, the solvent and the amylaceous material are introduced simultaneously in the form of a “slurry” type aqueous dispersion of amylaceous material.

In this method, the crosslinking agent, in this case the polyphosphate and more preferably sodium trimetaphosphate, which is introduced into the extruder and which will react with the amylaceous material only within the extruder, can either be added to the starch just before being introduced into the extruder (premix) or be introduced directly into the extruder already containing the amylaceous material to be crosslinked, possibly in the presence of a solvent. Preferably, the crosslinking agent is introduced directly into the extruder.

The crosslinking agent, in this case the polyphosphate, represents from 0.1% to 10% dry weight relative to the dry weight of amylaceous material used.

The zone of introduction of the polyphosphate and in particular its relative position in relation to the zone of introduction of the solvent (in particular water) is a critical parameter as regards the control of the competition between the destructuring of the amylaceous material and the crosslinking reaction between the nucleophilic groups possibly borne by the amylaceous material and at least one of the possible other ingredients.

It was in particular noticed that when the solvent (in particular water) was present in the extrudate in a proportion of at least 40% by weight of the extrudate at the time when the polyphosphate was introduced, products particularly advantageous in terms of their application were obtained. The extrudate refers to the whole of the material present in the extruder, hence at least the amylaceous material and the solvent, and indeed any other ingredients (such as plasticizers, lubricants and antimicrobials). Without wishing to be held to any one theory, the Applicant considers that these results are connected with the control of the phenomenon of destructuring of the amylaceous material and of its crystalline phase content, via the specific regulation of the solvent content.

In another version of the invention, a particularly preferred embodiment, the competition between these two mechanisms (destructuring-crosslinking) is managed, controlled by the further introduction of an alkaline catalyst which takes part in the destructuring of the starch. In this case, the extrusion stage a) further comprises the introduction of an alkaline catalyst into the extruder. It will then be preferred to introduce the crosslinking agent, in this case the polyphosphate, into the reaction medium before said alkaline catalyst (the polyphosphate is thus in its non-activated form): this makes it possible to disperse the polyphosphate in the material efficiently. The alkaline catalyst is then selected from alkali and alkaline earth metal oxides and hydroxides, in particular sodium hydroxide. The alkaline catalyst is preferably introduced into the extruder in the form of an aqueous solution or dispersion.

Preferably, the method according to the invention is a method for production of particles consisting of at least one crosslinked amylaceous material, comprising:
a) at least one stage of extrusion of at least one amylaceous material, in the presence of at least one crosslinking agent and
b) a granulation stage,
   characterized in that the crosslinking agent is a polyphosphate and in that the extrusion stage a) comprises:
i) a stage of introduction into an extruder of the amylaceous material into a first introduction zone of the extruder, of the crosslinking agent into a second zone, of at least one solvent into a third zone, and of the alkaline catalyst into a fourth zone, the second zone and the fourth zone being different, and
ii) a stage of kneading of the amylaceous material under strong shear in the presence in particular of the crosslinking agent.

According to a first embodiment of the method according to the invention, the first zone and second zone are identical. The introduction of the amylaceous material and the crosslinking agent can then be effected separately or as premix.

In this embodiment, the first zone is situated upstream of the third zone, itself situated upstream of the fourth zone.

According to a second embodiment of the method according to the invention, the second zone and third zone are identical. The introduction of the crosslinking agent and the solvent can then be effected separately or as premix.

In this embodiment, the first zone is situated upstream of the second zone, itself situated upstream of the fourth zone.

According to a third embodiment of the method according to the invention, the first, second and third zones are identical. The introduction of the amylaceous material, the crosslinking agent and the solvent can then be effected separately or as premix.

In this embodiment, the first zone is situated upstream of the fourth zone.

According to a fourth embodiment of the method according to the invention, the first zone and the fourth zone are identical. The introduction of the amylaceous material and of the alkaline catalyst can then be effected separately or as premix.

In this embodiment, the first zone is situated upstream of the third zone, itself situated upstream of the second zone.

The amylaceous material can be selected from the “granular starches”. Here, “granular starch” is understood to mean a natural starch, or one modified physically, chemically or enzymatically which has conserved within the granules of starch a semi-crystalline structure similar to that found in starch grains naturally present in the organs and storage tissues of higher plants, in particular in cereal grains, grains of leguminous plants, potato or manioc tubers, roots, bulbs, stems and fruit. This semi-crystalline state is essentially due to the macromolecules of amylopectin, one of the main constituents of starch. In the natural state, the starch grains exhibit a crystallinity level which varies from 15% to 45%, and which essentially depends on the botanical origin of the starch and any treatment that it has undergone. Granular starch, when placed under polarized light, exhibits a characteristic black cross, referred to as the Maltese cross, typical of the granular state.

According to the invention, the granular starch can derive from any botanical origin, including a granular starch rich in amylose or, conversely, rich in amylopectin (waxy). It can be natural starch from cereals such as wheat, maize, barley, amaranth, triticale, sorghum or rice, tubers such as potato or manioc, or leguminous plants such as the pea, mango bean and soya, and mixtures of such starches.

According to one embodiment, the granular starch is a starch hydrolyzed by the acid, oxidative or enzymatic route, or an oxidized starch. It can be a starch commonly called liquefied starch or a white dextrin.

According to another embodiment, it can also be a starch modified by physical-chemical means but having essentially conserved the structure of the starting natural starch, such as in particular esterified and/or etherified starches, in particular modified by acetylation, hydroxypropylation, cationization, crosslinking, phosphatation, or succinylation, or starches processed in an aqueous medium at low temperature (in English “annealing”). Preferably, the granular starch is a hydrolyzed, oxidized or modified natural starch, in particular from maize, wheat, peas or potato.

The granular starch generally has a content of less than 5% by mass of substances soluble at 20° C. in demineralized water. It is preferably almost insoluble in cold water.

According to a second embodiment, the amylaceous material can be a water-soluble starch, which can also derive from any botanical origin, including a water-soluble starch rich in amylose or, conversely, rich in amylopectin (waxy). This water-soluble starch can be introduced in partial or total replacement of the granular starch.

In the sense of the invention “water-soluble starch” is understood to mean any amylaceous compound exhibiting a soluble fraction in demineralized water at least equal to 5% by weight at 20° C. and with mechanical stirring for 24 hours. This soluble fraction is preferably greater than 20% by weight and in particular greater than 50% by weight. Of course, the water-soluble starch can be totally soluble in demineralized water (soluble fraction=100%).

Such water-soluble starches can be obtained by drum pregelatinization, by extruder pregelatinization, by atomization of an amylaceous suspension or solution, by precipitation by a non-solvent, by hydrothermal cooking, by chemical functionalization or other means. It is in particular a pregelatinized, extruded or atomized starch, a highly converted dextrin (also called yellow dextrin), a maltodextrin, a functionalized starch or any mixture of these products.

Pregelatinized starches can be obtained by hydrothermal gelatinization treatment of natural starches or modified starches, in particular by steam cooking, jet-cooker cooking, drum cooking, cooking in kneader/extruder systems then drying for example in an oven, with hot air on a fluidized bed, in a rotary drum, by atomization, by extrusion or by lyophilization. Such starches generally exhibit a solubility in demineralized water at 20° C. greater than 5% by weight and more generally of between 10% and 100% and a degree of crystallinity in starch lower than 15% (as diffraction intensity RX), generally lower than 5% and most often lower than 1%, or indeed zero. For example, the products manufactured and marketed by the Applicant under the trade name PREGEFLO® may be mentioned.

Highly converted dextrins are also among the amylaceous materials which can be used in the context of the invention. They can be prepared from natural or modified starches, by dextrinization in a low water content acidic medium. They can in particular be soluble white dextrins or yellow dextrins. For example, the products STABILYS® A 053 or TACKIDEX® C 072 manufactured and marketed by the Applicant may be mentioned. Such dextrins exhibit a solubility in demineralized water at 20° C., generally of between 10% and 95% by weight and a starch crystallinity lower than 15%, generally lower than 5%.

Maltodextrins and dehydrated glucose syrups are also suitable for the present invention. They can be obtained by acidic, oxidizing or enzymatic hydrolysis of starches in aqueous media. They can in particular exhibit a dextrose equivalent (DE) of between 0.5 and 40, preferably between 0.5 and 20 and better still between 0.5 and 12. Such maltodextrins or dehydrated glucose syrups are for example manufactured and marketed by the Applicant under the trade name GLUCIDEX® and exhibit a solubility in demineralized water at 20° C. generally of greater than 90%, or indeed close to 100%, and a starch crystallinity generally lower than 5% and usually almost zero.

The functionalized starches can be obtained from a natural or modified starch. The functionalization can for example be effected by esterification or etherification to a level sufficiently high to endow it with water solubility. Such functionalized starches have a soluble fraction, as defined above, greater than 5%, preferably greater than 10% and better still greater than 50%.

The functionalization can in particular be obtained by aqueous phase acetylation with acetic anhydride, by reaction with mixed anhydrides, by glue phase hydroxypropylation, by dry phase or glue phase cationization, by dry phase or glue phase anionization by phosphatation or succinylation. The water-soluble highly functionalized starches obtained can exhibit a degree of substitution of between 0.01 and 3, and better still between 0.05 and 1. Preferably, the reagents for modification or functionalization of the starch are of renewable origin.

According to another advantageous embodiment, the water-soluble starch is a water-soluble maize, wheat or pea starch, or a water-soluble derivative thereof. Further, it advantageously has a low water content, generally lower than 10%, preferably lower than 5%, in particular lower than 2.5% by weight, and ideally lower than 0.5%, or indeed lower than 0.2% by weight.

According to a third embodiment, the amylaceous component selected for the preparation of the composition is an organo-modified, preferably organo-soluble, starch, which can also be derived from any botanical origin, including an organo-modified, preferably organo-soluble, starch rich in amylose or, conversely, rich in amylopectin (waxy). This organo-soluble starch can be introduced in partial or total replacement of the granular starch or the water-soluble starch.

In the sense of the invention “organo-modified starch” is understood to mean any amylaceous component other than a granular starch or water-soluble starch according to the definitions given above. Preferably, this organo-modified starch is almost amorphous, that is to say exhibiting a degree of starch crystallinity lower than 5%, generally lower than 1% and in particular zero. It is also preferably “organo-soluble”, that is to say exhibiting at 20° C., a fraction at least equal to 5% by weight soluble in a solvent selected from ethanol, ethyl acetate, propyl acetate, butyl acetate, diethyl carbonate, propylene carbonate, dimethyl glutarate, triethyl citrate, dibasic esters, dimethyl sulfoxide (DMSO), dimethyl isosorbide, glycerol triacetate, isosorbide diacetate, isosorbide dioleate and the methyl esters of plant oils. This soluble fraction is preferably greater than 20% by weight and in particular greater than 50% by weight. Of course, the organo-soluble starch can be totally soluble in one or more of the solvents mentioned above (soluble fraction=100%).

The organo-modified starch can be utilized according to the invention in solid form, including having a low water content, namely lower than 10% by weight. It can in particular be lower than 5%, in particular lower than 2.5% by weight and ideally lower than 0.5%, or indeed lower than 0.2% by weight.

The organo-modified starch utilizable in the composition according to the invention can be prepared by functionalization of natural or modified starches such as those presented above. This functionalization can for example be effected by esterification or etherification to a sufficiently high degree to render it essentially amorphous and to endow it with insolubility in water and preferably solubility in one of the above organic solvents. Such functionalized starches exhibit a soluble fraction as defined above of greater than 5%, preferably greater than 10%, better still greater than 50%.

The functionalization can be obtained in particular by solvent phase acetylation with acetic anhydride, grafting, for example solvent phase or by reactive extrusion of acid anhydrides, mixed anhydrides, fatty acid chlorides, oligomers of caprolactones or lactides, glue phase hydroxypropylation and crosslinking, dry phase or glue phase cationization and crosslinking, anionization by phosphatation or succinylation, and dry phase or glue phase crosslinking, silylation, or butadiene telomerization.

These highly functionalized organo-modified, preferably organo-soluble, starches can in particular be acetates of starches, dextrins, maltodextrins dehydrated glucose syrups or fatty esters of these amylaceous materials (starches, dextrins, maltodextrins, dehydrated glucose syrups) with fatty chains of 4 to 22 carbons, these products together preferably exhibiting a degree of substitution (DS) of between 0.5 and 3.0, preferably between 0.8 and 2.8 and in particular between 1.0 and 2.7.

These can for example be hexanoates, octanoates, decanoates, laurates, palmitates, oleates and stearates of starch, dextrins, maltodextrins or dehydrated glucose syrups, in particular exhibiting a DS of between 0.8 and 2.8. According to another advantageous embodiment, the organo-modified starch is an organo-modified maize, wheat or pea starch or an organically modified derivative thereof.

In all cases, the dry material content of amylaceous material in the extruder is at least equal to 40%, preferably at least 50% and very preferably at least 60% by dry weight of the content of the extruder.

The amylaceous material or materials utilized according to the invention (alone, mixed or in combination as already indicated) can therefore be used with other ingredients. The latter can in particular be selected from cellulose, lignin, carboxymethylcellulose (CMC), hemicellulose, polyesters based on polybutylene succinate, polylactic acid or polyhydroxyalkanoates, thermoplastic polyurethanes, gluten, proteins and in particular pea proteins, polyamides, guar, xanthan, carrageenan, alginates, chitosah, cassia, tamarind, hemoglobin, gelatin, elastomers, lipids, triglycerides, saturated or unsaturated fatty acids, algae and macro-algae.

The second stage of the method according to the invention consists in granulating the extrudate leaving the extruder. This operation is performed by any available means enabling granulation.

The granulation is next followed by an optional grinding stage (stage c)), in particular a mechanical grinding stage on a solid, mechanical grinding after dispersion in a water or aqueous alcoholic solvent then followed by a stage of extraction of the solid (for example by lyophilization), or cryogenic grinding, the purpose of this treatment being to effect a decrease in the particle sizes of the granules derived from the previous stage.

In the sense of the invention, the granulation stage, apart from the granulation of the extrudate derived from the extrusion stage, can also include a grinding stage as described above.

According to one embodiment, the granulation in the sense of the invention can consist of a grinding stage as described above.

Finally, and optionally, the granules derived from stage b) or the ground particles derived from stage c) can be dispersed in water or an aqueous alcoholic solvent, preferably in water.

Advantageously, after grinding, particles of size between about 100 nm and 500 nm as determined by light scattering particle size analysis are obtained, which can easily be put into aqueous dispersion in water or an aqueous alcoholic solvent, and this with dry material contents of at least 20% starch dry weight, the dispersion thus obtained being entirely stable over time.

The invention also relates to the crosslinked starch particles obtained according to the method described above.

Another subject of the invention concerns the dispersion of the particles of crosslinked amylaceous material obtained according to the method described above and the resulting dispersions in water or in a solvent.

In other words, other subjects of the present invention are constituted of the granules resulting from the implementation of stages a) and b) of the method of the invention, the ground or granulated particles resulting from the implementation of stages a), b) and c) of the method of the invention and the dispersions in water or in an aqueous alcoholic solvent of granules or ground or granulated particles, said dispersions resulting from the implementation of stages a), b) and d) or a), b), c) and d) of the method of the invention.

Finally, a final subject consists in the use of the dispersions of the granules or the ground particles derived from the method according to the invention in the manufacture of wet films in general, in the manufacture of paper and in particular in the manufacture of coating colors, in the pharmaceuticals field as an active substance vehicle, in cosmetics, in agriculture and horticulture, in human and animal nutrition, and in the manufacture of mixtures with synthetic polymers.

### EXAMPLES

Specifically, different screw profiles can be used to control the specific mechanical energy transmitted to the material and thus to control the competition between destructuring of the starch and crosslinking reactions.

A screw profile is defined across the different zones which constitute said screw. Each zone (Z) is made up of a particular component (P) ensuring in particular transport or shear depending on a certain angle of the material which passes through it. Each zone is also associated with a particular temperature (T).

For the components, the following notations are used:

T: conveying components with various screw pitches
M: very dispersive mixing components with a very low shear component
C: includes all the components whose shear component is high, in other words all the shear components at 30, 45, 60 and 90° with direct pitch, and also 30, 45, 60° with reverse pitch and transport or mixing components with reverse pitch.

### EXAMPLE 1

This example illustrates the prior art, and corresponds in particular to the extrusion of a starch in the presence of glyoxal according to the protocol as described in the document EP 1 303 670 in its example 2.

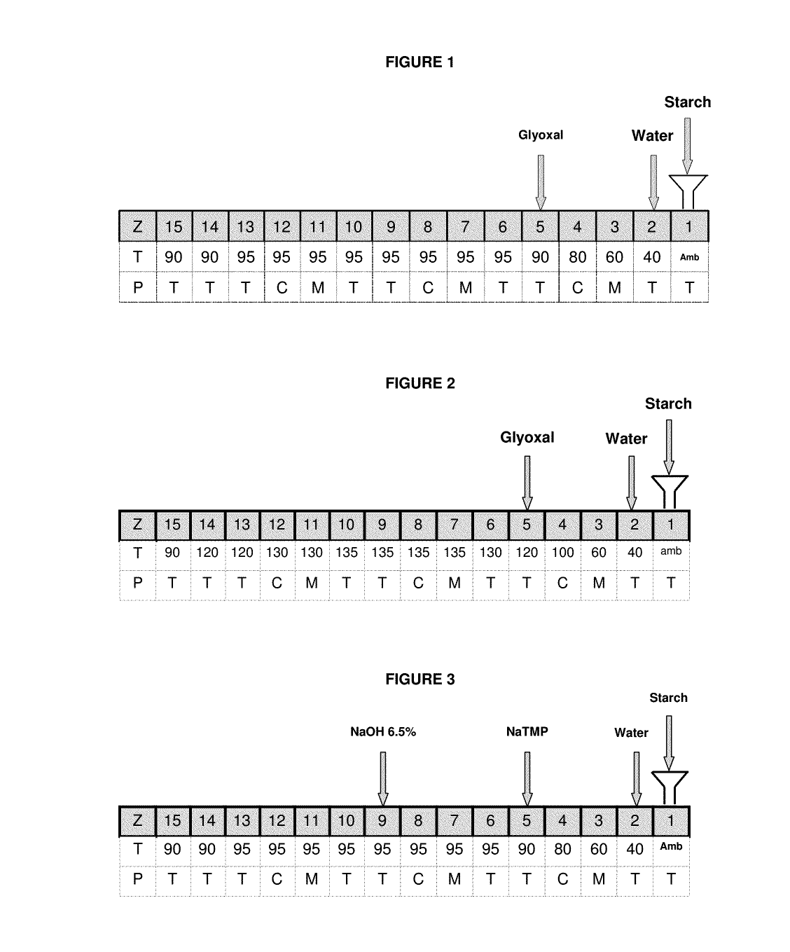

A mixture of natural maize starch (113 parts by weight the water content whereof equals 11.5%) and glycerol (17.9 parts by weight) is introduced into an extruder at a rate of 8.22 kg/h by means of a volumetric feeder. Said mixture is introduced into zone 1 of the extruder which has 15 zones, and exhibits a screw and temperature profile shown in FIG. 1. The screw speed is set at 500 revolutions/minute. Water (21 parts) is introduced at zone 2 at a rate of 0.6 kg/h by means of a piston pump. By means of the same device, glyoxal (1.9 parts) and water are introduced at zone 5, at a rate of 1.07 kg/h. At the end, the quantity of water in the extrudate is lower than 25% by weight and in particular 13% by weight of this was introduced before the addition of the crosslinking agent (glyoxal).

### EXAMPLE 2

This example illustrates the prior art, and corresponds in particular to the extrusion of a starch in the presence of glyoxal, according to the protocol as described in the document EP 1 303 670 in its example 2.

A mixture of natural maize starch (113 parts by weight the water content whereof equals 11.5%) and glycerol (17.9 parts by weight) is introduced into an extruder at a rate of 6.46 kg/h by means of a volumetric feeder. Said mixture is introduced into zone 1 of the extruder which has 15 zones, and exhibits a screw profile as shown in FIG. 2. The screw speed is set at 500 revolutions/minute. Water (20 parts including water from the natural starch) is introduced at zone 2, at a rate of 0.5 kg/h by means of a piston pump. By means of the same device, glyoxal (1 part) and water are introduced at zone 5, at a rate of 1.39 kg/h. At the end, the quantity of water in the extrudate is lower than 31% by weight and in particular 14% by weight of this was introduced before the addition of the crosslinking agent (glyoxal).

### EXAMPLE 3

This example illustrates the invention. Natural maize starch (113 parts by weight the water content whereof equals 12%) is introduced into an extruder at a rate of 4.94 kg/h by means of a volumetric feeder. It is introduced into zone 1 of the extruder which has 15 zones, and exhibits a screw profile as shown in FIG. 3. The screw speed is set at 500 revolutions/minute. Water (170.4 parts including water from the natural starch) is introduced at zone 2, at a rate of 0.5 kg/h by means of a piston pump. By means of the same device, sodium trimetaphosphate (2.3 parts) in solution is introduced at zone 5, at a rate of 0.1 kg/h. At zone 9, a solution of sodium hydroxide (0.74 parts) is introduced at a rate of 0.032 kg/h. At the end, the quantity of water in the extrudate is equal to 65.5% by weight and in particular 60% by weight of this was introduced before the addition of the crosslinking agent (trimetaphosphate).

### EXAMPLE 4

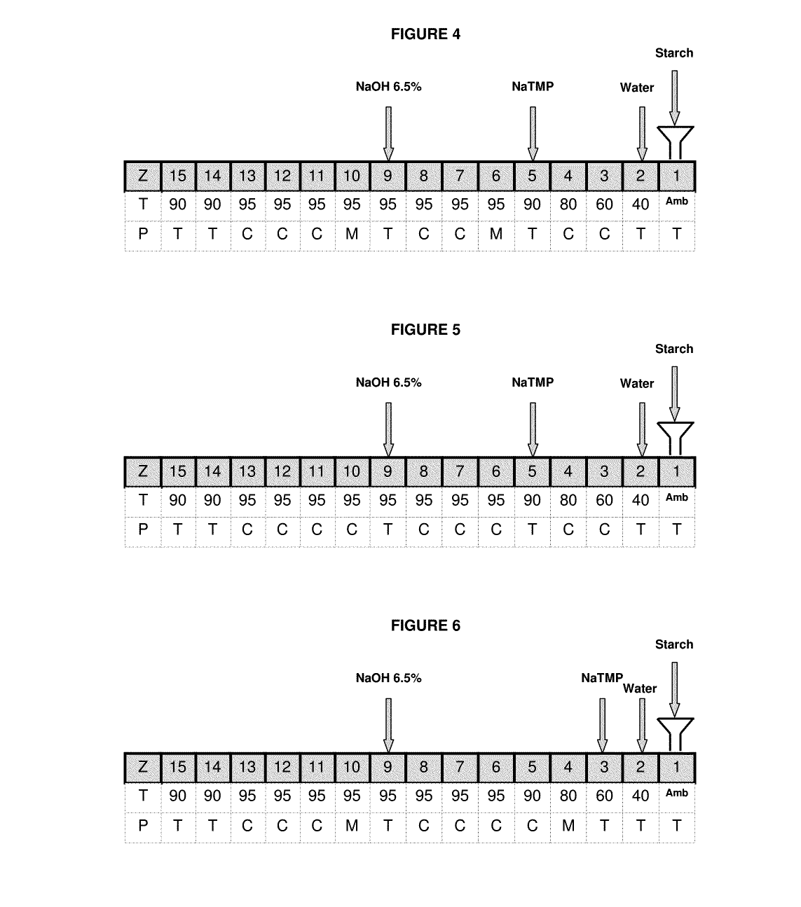

This example illustrates the invention; it is identical to the previous one, with the difference that the extruder has a profile as shown in FIG. 4.

### EXAMPLE 5

This example illustrates the invention; it is identical to example 3, with the difference that the extruder has a profile as shown in FIG. 5.

### EXAMPLE 6

This example also illustrates the invention. Natural maize starch (113 parts by weight the water content whereof equals 12%) is introduced into an extruder at a rate of 4.94 kg/h by means of a volumetric feeder. It is introduced into zone 1 of the extruder which has 15 zones, and exhibits a screw profile as shown in FIG. 6. The screw speed is set at 500 revolutions/minute. Water (170.4 parts including water from the natural starch) is introduced at zone 2, at a rate of 0.5 kg/h by means of a piston pump. By means of the same device, sodium trimetaphosphate (2.3 parts in solution is introduced at zone 3, at a rate of 0.1 kg/h. At zone 9, a solution of sodium hydroxide (0.74 parts) is introduced at a rate of 0.032 kg/h. At the end, the quantity of water in the extrudate is equal to 65.5% by weight and in particular 60% by weight of this was introduced before the addition of the crosslinking agent.

### EXAMPLE 7

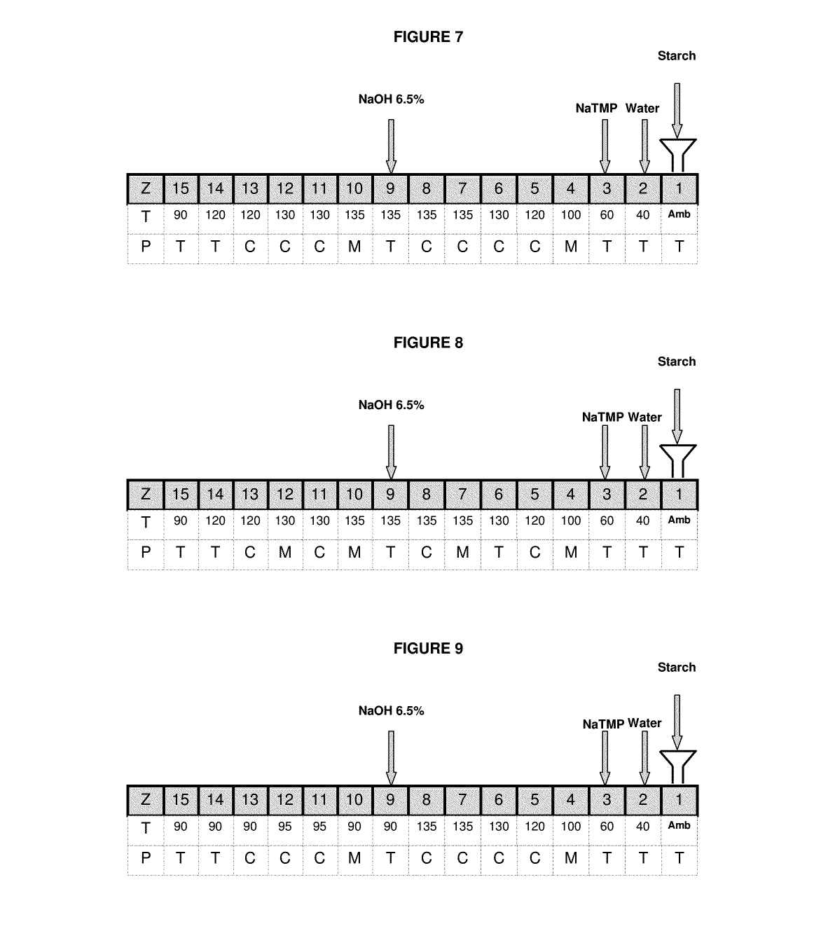

This example illustrates the invention; it is identical to the previous one, with the difference that the extruder has a profile as shown in FIG. 7.

### EXAMPLE 8

This example illustrates the invention; it is identical to the previous one, with the difference that the extruder has a profile as shown in FIG. 8.

### EXAMPLE 9

This example also illustrates the invention; it is identical to the previous one, with the difference that the extruder has a profile as shown in FIG. 9.

### EXAMPLE 10

This example illustrates the invention; it is identical to example 3, but the screw speed is set at 250 revolutions per minute.

### EXAMPLE 11

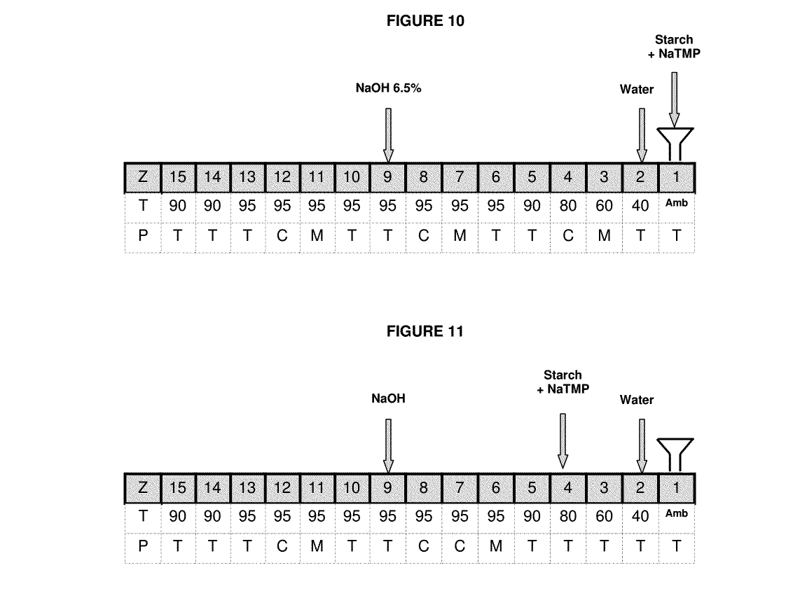

This example illustrates the invention; it is identical to example 3, with the difference that sodium trimetaphosphate is premixed with the starch and introduced in this form at zone 1, while the soda solution is introduced in zone 9 (see FIG. 10).

### EXAMPLE 12

This example illustrates the invention; it is identical to example 11, with the difference that water is introduced before the mixing of the starch and sodium trimetaphosphate (see FIG. 11).

### EXAMPLE 13

This example illustrates the invention; it is identical to example 3, with the difference that the starch is a potato starch.

### EXAMPLE 14

This example illustrates the invention; it is identical to example 3, with the difference that the starch is a rice starch.

### EXAMPLE 15

This example illustrates the invention; it is identical to example 3, with the difference that the starch is a pea starch.

### EXAMPLE 16

This example illustrates the invention; it is identical to example 3, with the difference that the starch is an anionic maize starch.

### EXAMPLE 17

This example illustrates the invention; it is identical to example 3, with the difference that the starch is a cationic maize starch.

### EXAMPLE 18

This example illustrates the invention; it is identical to example 3, with the difference that the starch is a hydroxy-propylated starch.

### EXAMPLE 19

This example illustrates the invention; it is identical to example 3, with the difference that the compound introduced is a mixture of waxy starch and corn flour.

All of the following experiments were performed on an extruder of the Leistritz ZSE 27 maxx type having an L/D ratio=60, and possessing 15 zones.

The powders are introduced into the extruder by means of Schlenck solid gravimetric feeders of the Proflex type.

The liquids are introduced into the extruder by means of liquid gravimetric feeders of the Brabender type when the flow rates are greater than 1 kg/h. When the liquid flow rates are lower than 1 kg/h, gravimetric microfeeders are used.

The temperature profile utilized is given in table 1 below.

TABLE 1

| | zone | | | | | | | | | | | | | | |
|---|---|---|---|---|---|---|---|---|---|---|---|---|---|---|---|
| | 15 | 14 | 13 | 12 | 11 | 10 | 9 | 8 | 7 | 6 | 5 | 4 | 3 | 2 | 1 |
| temperature (° C.) | 105 | 105 | 95 | 95 | 95 | 95 | 95 | 95 | 95 | 95 | 95 | 95 | 95 | 80 | 60 |

The screw profiles used are made up of the following components: (M=mixing component, C=shear component, T=transport component)

TABLE 2

| | zone | | | | | | | | | | | | | | |
|---|---|---|---|---|---|---|---|---|---|---|---|---|---|---|---|
| type of profile | 15 | 14 | 13 | 12 | 11 | 10 | 9 | 8 | 7 | 6 | 5 | 4 | 3 | 2 | 1 |
| 1 | T | T | T | T | T | T | T/M | M/C | M/C | M/T | T/C | T/C | M/T | T | T |
| 2 | T | T | T/M | M | M/T | C | C | M | T | T/C | M/C | T | T | T | T |
| 3 | T | T | M | M | M/T | C | M/C | M/T | T | C/M | M/C | T | T | T | T |
| 4 | T | C | T/C | T | T/C | T | T/C | T/C | C | C | T | T | T | T | T |

The screw speed for all the experiments enumerated in the table below is set at 400 revolutions/min.

In all the examples the reagents are introduced separately except for experiment 26 in which a premix containing 10 kg of potato starch, 13.9 kg water and 543 g of sodium trimetaphosphate (STMP) and 10 g of Irgasan®.

Irgasan® is an antimicrobial agent.

Eurylon® is maize starch with a high amylose content.

Experiments 2 to 30 are according to the invention.

Experiment 1 is not in accordance with the invention.

| | Zone 1 | | | | | | | | | | | | | | | | | | | | |
|---|---|---|---|---|---|---|---|---|---|---|---|---|---|---|---|---|---|---|---|---|---|
| | | Flow | | | | | | | Zone 3 | | | | | Zone 6 or 7 | | | | | | | |
| | | (kg/h) | | | Flow | Irgasan ® | | | | | Flow | H2O/ | Flow | | | Flow | Intro | | Flow | Intro | Screw |
| Expt. No. | Starch | (corn) | Reagent | % (s/s) | (kg/h) | (kg/h) | H2O | Flow (kg/h) | Reagent | % (s/s) | (kg/h) | reagent | (kg/h) | Reagent | % (s/s) | (kg/h) | zone | H2O | (kg/h) | zone | profile |
| 1 | waxy | 10.00 | | | | 0.01 | | | glycerol | 18.2 | 1.6 | H₂O | 4.27 | Glyoxal | 2.5 | 0.22 | 6 | yes | 1.6 | 6 | 1 |
| 2 | waxy | 10.00 | STMP | 0.1 | 0.01 | | | | | | | H₂O | 6.25 | NaOH | 0.0 | 0.004 | 7 | yes | 1 | 7 | 1 |
| 3 | waxy | 10.00 | STMP | 0.2 | 0.01 | | | | | | | H₂O | 6.25 | NaOH | 0.1 | 0.007 | 7 | yes | 1 | 7 | 1 |
| 4 | waxy | 10.00 | STMP | 0.3 | 0.029 | | | | | | | H₂O | 6.25 | NaOH | 0.2 | 0.014 | 7 | yes | 1 | 7 | 1 |
| 5 | waxy | 10.00 | STMP | 0.7 | 0.058 | | | | | | | H₂O | 6.25 | NaOH | 0.3 | 0.027 | 7 | yes | 1 | 7 | 1 |
| 6 | waxy | 10.00 | STMP | 1.3 | 0.016 | | | | | | | H₂O | 6.25 | NaOH | 0.6 | 0.055 | 7 | yes | 1 | 7 | 1 |
| 7 | waxy | 10.00 | NaOH | 1.3 | 0.11 | | | | | | | H₂O | 5.25 | STMP | 2.6 | 0.233 | 7 | yes | 2 | 7 | 1 |
| 8 | waxy | 10.00 | STMP | 5.3 | 0.465 | 0.01 | | | | | | H₂O | 5.9 | NaOH | 2.5 | 0.22 | 6 | yes | 1.35 | 6 | 1 |
| 9 | waxy | 10.00 | STMP | 10.0 | 0.88 | 0.01 | | | | | | H₂O | 4.1 | NaOH | 4.8 | 0.42 | 6 | yes | 2.6 | 6 | 1 |
| 10 | waxy | 10.00 | STMP | 2.7 | 0.235 | 0.01 | | | | | | H₂O | 4.35 | NaOH | 1.3 | 0.11 | 6 | yes | 2.6 | 6 | 1 |
| 11 | waxy | 12.00 | STMP | 5.3 | 0.555 | 0.01 | | | | | | H₂O | 4.50 | NaOH | 2.6 | 0.275 | 6 | yes | 2.1 | 6 | 1 |
| 12 | waxy | 15.00 | | | | | | | STMP | 1.0 | 0.13 | H₂O | 9.88 | NaOH | 0.5 | 0.062 | 7 | yes | 1 | 7 | 1 |
| 13 | waxy | 15.00 | | | | | | | STMP | 0.6 | 0.08 | H₂O | 9.88 | NaOH | 0.3 | 0.41 | 7 | yes | 1 | 7 | 1 |
| 14 | waxy | 15.00 | | | | | | | STMP | 0.3 | 0.04 | H₂O | 9.88 | NaOH | 0.2 | 0.21 | 7 | yes | 1 | 7 | 1 |
| 15 | waxy | 15.00 | | | | | | | STMP | 1.0 | 0.13 | H₂O | 9.88 | NaOH | 0.5 | 0.62 | 7 | yes | 1 | 7 | 2 |
| 16 | waxy | 15.00 | | | | | | | STMP | 0.6 | 0.08 | H₂O | 9.88 | NaOH | 0.3 | 0.041 | 7 | yes | 1 | 7 | 2 |
| 17 | waxy | 15.00 | | | | | | | STMP | 0.3 | 0.04 | H₂O | 9.88 | NaOH | 0.2 | 0.021 | 7 | yes | 1 | 7 | 2 |
| 18 | waxy | 15.00 | | | | | | | STMP | 1.0 | 0.13 | H₂O | 9.88 | NaOH | 0.5 | 0.62 | 7 | yes | 1 | 7 | 3 |
| 19 | waxy | 15.00 | | | | | | | STMP | 0.6 | 0.08 | H₂O | 9.88 | NaOH | 0.3 | 0.041 | 7 | yes | 1 | 7 | 3 |
| 20 | waxy | 15.00 | | | | | | | STMP | 0.3 | 0.04 | H₂O | 9.88 | NaOH | 0.2 | 0.21 | 7 | yes | 1 | 7 | 3 |
| 21 | waxy | 15.00 | | | | | | | STMP | 1.0 | 0.13 | H₂O | 9.88 | NaOH | 0.5 | 0.062 | 7 | yes | 1 | 7 | 4 |
| 22 | waxy | 15.00 | | | | | | | STMP | 0.6 | 0.08 | H₂O | 9.88 | NaOH | 0.3 | 0.041 | 7 | yes | 1 | 7 | 4 |
| 23 | waxy | 15.00 | | | | | | | STMP | 0.3 | 0.04 | H₂O | 9.88 | NaOH | 0.2 | 0.021 | 7 | yes | 1 | 7 | 4 |
| 24 | waxy | 15.00 | | | | | | | STMP | 9.9 | 1.31 | H₂O | 9.88 | NaOH | 4.7 | 0.62 | 7 | yes | 1 | 7 | 4 |
| 25 | waxy | 15.00 | | | | | | | STMP | 0.1 | 0.01 | H₂O | 9.88 | NaOH | 0.0 | 0.006 | 7 | yes | 1 | 7 | 4 |
| 26 | corn flour | 10.00 | STMP | 5.2 | 0.435 | 0.01 | yes | 13.93 | | | | | | NaOH | 2.5 | 0.205 | 6 | yes | 2.6 | 6 | 1 |
| 27 | Eurylon ® | 10.00 | STMP | 5.2 | 0.465 | 0.01 | | | | | | H₂O | 4.55 | NaOH | 2.5 | 0.22 | 6 | yes | 2.6 | 6 | 1 |
| 28 | pea | 15.00 | | | | | | | | 0.6 | 0.08 | H₂O | 9.88 | NaOH | 0.3 | 0.041 | 7 | yes | 1 | 7 | 1 |
| 29 | maize | 15.00 | | | | | | | | 0.6 | 0.08 | H₂O | 9.88 | NaOH | 0.3 | 0.041 | 7 | yes | 1 | 7 | 1 |
| 30 | cationic | 15.00 | | | | | | | | 0.6 | 0.08 | H₂O | 9.88 | NaOH | 0.3 | 0.041 | 7 | yes | 1 | 7 | 1 |
| | starch | | | | | | | | | | | | | | | | | | | | |

Characterization of the Degree of Destructuring:

In order to characterize the presence or absence of raw or swollen grains, rod cross-section observations by microscopy are made in polarized light by means of a LEICA microscope (model: Leitz DMRB) with ×10 and ×20 objectives.

In order to do this, a part of the rods thus obtained is taken, and a cross-section of the rod is created directly by means of a razor blade. In the case of rods exhibiting too great a hardness, the cross-sections (of about 10 μm) can be created by means of a LEICA microtome (model: Jung RM 2055). In this case, pieces of rods of length 2 cm are cut, then fixed on a support and enveloped in a LEICA histological resin. The cross-sections thus obtained are then placed on a slide in a water and/or glycerol solution maintained at 45° C. on a heated bench. Finally, the preparation is covered with a cover slip for observation.

The 2 magnifications ×10 and ×20 make it possible to assess the presence or absence of non-destructured starch grains.

In experiment 1 not in accordance with the invention, a continuous matrix of starch is observed in which the grains are no longer visible, as they have been totally destructured. Conversely, in experiments 2 to 30 according to the invention, polarization crosses (Maltese cross—polarization in form of cross) are very distinctly observed in the presence of uncooked grains (crystalline phase still present). In this manner, we confirm the value of the invention, namely the control of the level of destructuring of the starch involved depending on the method employed.

1. A method for production of particles consisting of at least one amylaceous material, comprising:
a) at least one stage of extrusion of at least one amylaceous material, in the presence of at least one crosslinking agent,
b) a granulation stage,
c) possibly a grinding stage, and
d) possibly a stage of dispersion in a solvent
and characterized in that the crosslinking agent is a polyphosphate and in that the extrusion stage a) is performed by introduction of the amylaceous material, the polyphosphate and a solvent into the extruder.

2. The method as claimed in claim 1, characterized in that stage a) is performed at a temperature at least equal to 40° C., preferably at least 50° C. and very preferably at least 60° C.

3. The method as claimed in claim 1, characterized in that the polyphosphate represents from 0.1% to 10% dry weight relative to the dry weight of amylaceous material.

4. The method as claimed in claim 1, characterized in that the solvent represents at least 40% by weight of the extrudate at the time when the polyphosphate is introduced.

5. The method as claimed in claim 1, characterized in that the extrusion stage a) is performed with introduction of an alkaline catalyst into the extruder.

6. The method as claimed in claim 5, characterized in that the polyphosphate is introduced into the extruder before the alkaline catalyst.

7. The method as claimed in claim 5, characterized in that the alkaline catalyst is selected from alkali and alkaline earth metal oxides and hydroxides.

8. The method as claimed in claim 1, characterized in that the solvent of the extrusion stage a) is selected from water and aqueous alcoholic solvents, and is preferably water.

9. The method as claimed in claim 1, characterized in that the polyphosphate is sodium trimetaphosphate.

10. Granules obtained by the method as claimed in claim 1.

11. Ground particles obtained by the method as claimed in claim 1.

12. Dispersions in water or in an aqueous alcoholic solvent of granules or of ground particles obtained by the method as claimed in claim 1.

13. A method of preparing a composition comprising adding to a composition the granules as claimed in claim 10 wherein the composition is selected from the group consisting of a wet film, in paper, a coating color, an active substance vehicle for a medicament, a cosmetic, an agriculture or a horticulture composition, a human or an animal nutritional composition and a mixture with synthetic polymers.

14. A method of preparing a composition comprising adding to a composition the ground particles as claimed in claim 11, wherein said composition is selected from the group consisting of a wet film, paper, a coating color, an active substance vehicle for a medicament, a cosmetic, an agriculture or a horticulture composition, a human or an animal nutritional composition, and a mixture with synthetic polymers.

15. A method of preparing a composition comprising adding to a composition the dispersions as claimed in claim 12, wherein said composition is selected from the group consisting of a wet film, paper, a coating color, an active substance vehicle for a medicament, a cosmetic, an agriculture or a horticulture composition, a human or an animal nutritional composition, and a mixture with synthetic polymers.

16. A composition comprising the granules as claimed in claim 10, wherein said composition is selected from the group consisting of a wet film, paper, a coating color, an active substance vehicle for a medicament, a cosmetic, an agriculture or a horticulture composition, a human or an animal nutritional composition, and a mixture with synthetic polymers.

17. A composition comprising the ground particles as claimed in claim 11, wherein said composition is selected from the group consisting of a wet film, paper, a coating color, an active substance vehicle for a medicament, a cosmetic, an agriculture or a horticulture composition, a human or an animal nutritional composition, and a mixture with synthetic polymers.

18. A composition comprising the dispersions as claimed in claim 12, wherein said composition is selected from the group consisting of a wet film, paper, a coating color, an active substance vehicle for a medicament, a cosmetic, an agriculture or a horticulture composition, a human or an animal nutritional composition, and a mixture with synthetic polymers.
